(19) United States
(12) Patent Application Publication
(10) Pub. No.: US 2016/0030702 A1
(43) Pub. Date: Feb. 4, 2016

(54) AUDIO PLAY DEVICE AND METHOD THEREOF AND DISPLAY DEVICE INCLUDING AUDIO PLAY DEVICE

(71) Applicants: BOE TECHNOLOGY GROUP CO., LTD., Beijing (CN); BEIJING BOE MULTIMEDIA TECHNOLOGY CO., LTD., Beijing (CN)

(72) Inventor: Shuai YANG, Beijing (CN)

(21) Appl. No.: 14/430,023

(22) PCT Filed: May 21, 2014
(86) PCT No.: PCT/CN2014/078011

§ 371 Date: Mar. 20, 2015

(30) Foreign Application Priority Data
Dec. 31, 2013 (CN) 201310752754.8

(51) Int. Cl. A61M 21/02; A61B 19/00; A61B 5/00

(52) U.S. Cl. CPC A61M 21/02; A61B 5/4809; A61B 19/5212; A61M 2021/0027

(57) ABSTRACT

The present invention provides an audio play device for inducing a user to fall asleep and a method thereof. The audio play device comprises: an identifying module for identifying a user's state; a frequency adjusting module for adjusting the frequency of an initial audio signal according to the identified user's state to generate a first audio signal and a second audio signal having frequency difference, the initial audio signal being the audio signal from a program source; a generation module for generating a first sound wave according to the first audio signal and generating a second sound wave according to the second audio signal; and a playing module for causing the first sound wave and the second wave to enter left ear and right ear of the user respectively at the same time.

### FIELD OF THE INVENTION

The present invention relates to an audio play device and a method thereof, and particularly relates to an audio play device for inducing a user to fall asleep and a method thereof, and a display device including the audio play device.

### BACKGROUND OF THE INVENTION

In the modern society, people suffer from reduced sleep time and reduced quality of sleep due to an increasing work pressure. In this case, a variety of appliances with a function of inducing people to fall asleep have been developed. Among them, a TV set with a function of inducing people to fall asleep is included.

According to the prior art, to implement the TV set's function of inducing people to fall asleep, people are induced to fall asleep generally by playing fixed pictures or music with hypnotic effect.

However, in the prior art there are at least the following problems: the above-mentioned way of inducing people to fall asleep will affect normal use of the TV set, so that the TV set cannot play an original TV program; in addition, in the prior art, an optimum mode of inducing people to fall asleep will not be selected based on the current state of the user so that the result of inducing people to fall asleep is not ideal.

At present, determined brain electrical activity modes are mainly contained in frequencies lower than 30 Hz. There are four brain electrical activity modes: β mode in which brain wave having frequency of 15 Hz to 30 Hz is dominant, the brain being in a state of highly awake and active thinking (i.e. an exciting state); α mode in which brain wave having frequency of 8 Hz to 14 Hz is dominant, the brain being in a state of relaxation but still conscious (i.e. an tired state); θ mode in which brain wave having frequency of 4 Hz to 7 Hz is dominant, the brain being in a state of subconscious (i.e. an sleepy state); and δ mode in which brain wave having frequency of 0.5 Hz to 3 Hz is dominant, the brain being in a state of deep sleep (i.e. a sleeping state). It can be observed via an electroencephalogram (EEG) that the above-mentioned four kinds of brain waves exist at the same time, but the proportions which various brain waves account for are different under different brain electrical activity modes.

Binaural beats refer to that when two sound waves being slightly different in frequency but carrying same information are respectively received in both ears at the same time, the brain will generate a corresponding rhythmic neuroelectricity activity, i.e. generate brain waves of the corresponding mode. For example, a 10 Hz brain wave will be generated when a 420 Hz sound wave is heard in a left ear and a 410 Hz sound wave is heard in a right ear. For producing the binaural beats, the carrier frequency should be lower than 1500 Hz and the frequency difference between sound waves heard in the left and right ears is not higher than 30 Hz; otherwise, two different sounds will be heard in both ears distinctly.

In the Chinese patent application CN 200880021657.9, disclosed is a sound and light machine capable of producing sound and light pulses having brain wave frequencies. The LEDs of the sound and light machine are configured to be placed in front of the user's left and right eyes respectively, and programmed to emit light pulse having a frequency that induces the user into a particular consciousness state, and speakers of the sound and light machine are configured to play different tones into the user's right and left ears to produce binaural beats that match the frequency of the pulse. The sound and light machine can induce the user into a particular consciousness state. The entire of that patent application is incorporated herein by reference. However, in the art disclosed in the Chinese patent application CN 200880021657.9, the user's current state was not considered. In other words, the sound and light machine will not select an optimum inducing mode according to the user's state, just like the TV set with a function of inducing people to fall asleep.

### SUMMARY OF THE INVENTION

According to the above-mentioned principle, the present invention provides a way of inducing a user to fall asleep different from the prior art in which the user's brain electrical activity mode is changed by using the binaural beats, so that the user can be induced to fall asleep while enjoyment of the user on audio and video entertainment is not affect. In addition, an optimum mode can be selected based on the user's state so that the user can fall asleep more effectively in a short time.

According to one aspect of the present invention, an audio play method for inducing a user to fall asleep is provided, including steps of identifying the user's state; adjusting frequency of an initial audio signal according to the identified user's state to generate a first audio signal and a second audio signal having frequency difference; generating a first sound wave according to the first audio signal and generating a second sound wave according to the second audio signal; and causing the first sound wave and the second sound wave to enter left ear and right ear of the user respectively at the same time.

According to an embodiment of the present invention, the step of identifying a user's state may include: acquiring the user's physiological information; and identifying the user's state according to the acquired physiological information.

According to an embodiment of the present invention, the user's state may, according to the acquired physiological information of the user, be identified as one of the following states: exciting state, tired state, sleepy state and sleeping state.

According to an embodiment of the present invention, the user's physiological information may include the user's brain wave information; the user's state may be identified as the exciting state when brain wave having frequency of 15 Hz to 30 Hz is dominant; the user's state may be identified as the tired state when brain wave having frequency of 8 Hz to 14 Hz is dominant; the user's state may be identified as the sleepy state when brain wave having frequency of 4 Hz to 7 Hz is dominant; and the user's state can be identified as the sleeping state when brain wave having frequency of 0.5 Hz to 3 Hz is dominant. Alternatively, a user's physiological information may include at least one of temperature information, image information and pulse information.

According to an embodiment of the present invention, in the step of adjusting the frequency of the initial audio signal according to the user's state to generate the first audio signal and the second audio signal, the frequency difference between the first audio signal and the second audio signal may be caused to be in a range from 8 Hz to 14 Hz when the user's state is identified as the exiting state; the frequency difference between the first audio signal and the second audio signal may be caused to be in a range from 4 Hz to 7 Hz when the user's state is identified as the tired state; and the frequency difference between the first audio signal and the second audio signal may be caused to be in a range from 0.5 Hz to 3 Hz when the user's state is identified as the sleepy state or sleeping state.

According to an embodiment of the present invention, one of the first audio signal and the second audio signal may be the same as the initial audio signal.

According to an embodiment of the present invention, the step of generating the first sound wave according to the first audio signal and generating the second sound wave according to the second audio signal may include: performing pulse width modulation on the first audio signal and the second audio signal to form a first pulse width modulation signal and a second pulse width modulation signal respectively; and amplifying the first pulse width modulation signal and the second pulse width modulation signal to generate the first sound wave and the second wave, respectively.

According to an embodiment of the present invention, the first audio signal and the second audio signal may be generated simultaneously.

According to an embodiment of the present invention, the first sound wave and the second sound wave may be caused to enter the user's left and right ears respectively at the same time by using any one of earphones, directional speakers and directional sound boxes.

According to another aspect of the present invention, an audio play device for inducing a user to fall asleep is provided. The audio play device includes: an identifying module for identifying a user's state; a frequency adjusting module for adjusting frequency of an initial audio signal according to the identified user's state to generate a first audio signal and a second audio signal having frequency difference; a generation module for generating a first sound wave according to the first audio signal and generating a second sound wave according to the second audio signal; and a playing module for causing the first sound wave and the second sound wave to enter left ear and right ear of the user respectively at the same time.

According to an embodiment of the present invention, the identifying module may include: an acquiring module for acquiring the user's physiological information; and a judging module for identifying the user's state according to the acquired physiological information.

According to an embodiment of the present invention, the judging module may, according to the acquired physiological information of the user, identify the user's state as one of the following states: exciting state, tired state, sleepy state, and sleeping state.

According to an embodiment of the present invention, the acquiring module may include a brain wave acquiring device for acquiring the user's brain wave information; the judging module may identify the user's state as the exciting state when brain wave having frequency of 15 Hz to 30 Hz is dominant; the judging module may identify the user's state as the tired state when brain wave having frequency of 8 Hz to 14 Hz is dominant; the judging module may identify the user's state as the sleepy state when brain wave having frequency of 4 Hz to 7 Hz is dominant; and the judging module may identify the user's state as the sleeping state when brain wave having frequency of 0.5 Hz to 3 Hz is dominant. Alternatively, the acquiring module may include at least one of an infrared temperature sensor, an image acquiring device and a pulse detecting instrument to acquire the temperature information, image information and/or pulse information.

According to an embodiment of the present invention, the frequency adjusting module may cause the frequency difference between the first audio signal and the second audio signal to be in a range from 8 Hz to 14 Hz when the user's state is identified as the exiting state; the frequency adjusting module may cause the frequency difference between the first audio signal and the second audio signal to be in a range from 4 Hz to 7 Hz when the user's state is identified as the tired state; and the frequency adjusting module may cause the frequency difference between the first audio signal and the second audio signal to be in a range from 0.5 Hz to 3 Hz when the user's state is identified as the sleepy state or sleeping state.

According to an embodiment of the present invention, the frequency adjusting module may keep one of the first audio signal and the second audio signal being the same as the initial audio signal.

According to an embodiment of the present invention, the frequency adjusting module may include a first adjusting submodule and a second adjusting submodule for adjusting the frequency of the initial audio signal according to the user's state, so as to generate the first audio signal and the second audio signal respectively, and the generation module may include a first generating submodule and a second generating submodule for generating the first sound wave and the second sound wave respectively according to the first audio signal and the second audio signal.

According to an embodiment of the present invention, each of the first generating submodule and the second generating submodule may include: a pulse width modulation module for performing pulse width modulation on the first audio signal or the second audio signal to form a first pulse width modulation signal or a second pulse width modulation signal; and a power amplifying module for amplifying the first pulse width modulation signal or the second pulse width modulation signal to generate the first sound wave and the second wave.

According to an embodiment of the present invention, the generation module may further include a synchronization module for controlling the first audio signal and the second audio signal to be generated simultaneously.

According to an embodiment of the present invention, the playing module may include one of earphones, directional speakers and directional sound boxes.

According to another aspect of the present invention, a display device is further provided, including the audio play device according to the present invention for inducing a user to fall asleep.

According to the technical solutions of the present invention, a user can be induced to fall asleep while enjoyment of the user on audio and video entertainment is not affected. In addition, according to the technical solutions of the present invention, an optimum mode can be selected based on the user's state so that the user can fall asleep more effectively in a short time

### BRIEF DESCRIPTION OF THE DRAWINGS

FIG. 1 is a schematic block diagram of an audio play device according to an embodiment of the present invention;

FIG. 2 is a schematic block diagram of an identifying module included in the audio play device according to an embodiment of the present invention;

FIG. 3A is a schematic block diagram of a frequency adjusting module included in the audio play device according to an embodiment of the present invention;

FIG. 3B is a schematic block diagram of an adjusting submodule included in the frequency adjusting module;

FIG. 4A is a schematic block diagram of a generation module included in the audio play device according to an embodiment of the present invention;

FIG. 4B is a schematic block diagram of a generating submodule included in the generation module; and

FIG. 5 is a schematic flowchart of an audio play method according to an embodiment of the present invention.

### DETAILED DESCRIPTION OF THE EMBODIMENTS

To make the person skilled in the art better understand the technical solutions of the present invention, embodiments of the present invention will be further described below in details in conjunction with the accompanying drawings.

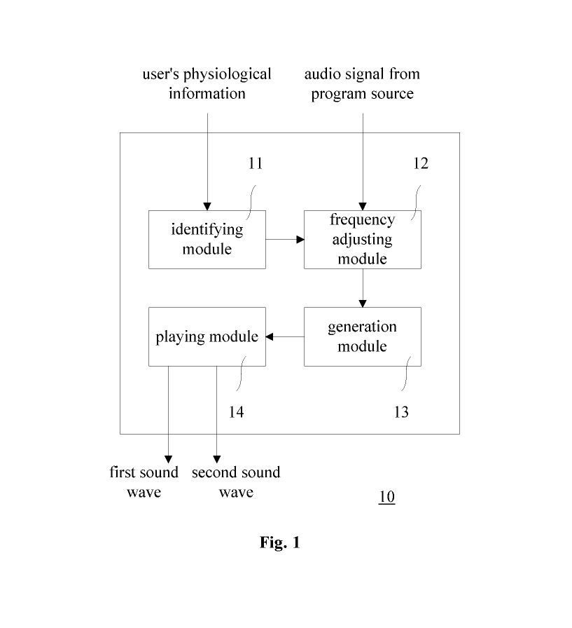

FIG. 1 is a schematic block diagram of an audio play device 10 according to an embodiment of the present invention.

Referring to FIG. 1, the audio play device 10 according to the embodiment of the present invention may include an identifying module 11, a frequency adjusting module 12, a generation module 13 and a playing module 14. The identifying module 11 is used for identifying a user's state. The frequency adjusting module 12 is used for adjusting frequency of an initial audio signal according to the identified user's state to generate a first audio signal and a second audio signal having frequency difference, wherein the initial audio signal may be an audio signal from a program source. The generation module 13 is used for generating a first sound wave according to the first audio signal and generating a second sound wave according to the second audio signal. The playing module 14 causes the first sound wave and the second sound wave to enter the left ear and right ear of the user respectively at the same time.

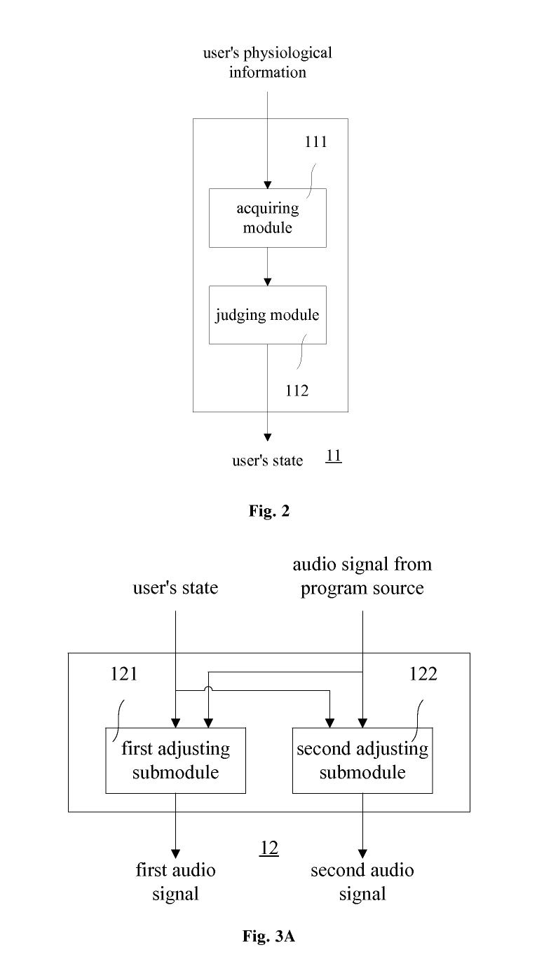

Next, each of the above-mentioned modules will be described in details with reference to FIG. 2 to FIG. 4B.

FIG. 2 is a schematic block diagram of the identifying module 11 included in the audio play device 10.

Referring to FIG. 2, the identifying module 11 may include an acquiring module 111 for acquiring a user's physiological information and a judging module 112 for identifying the user's state according to the acquired physiological information.

The acquiring module 111 may include at least one of a user wearable device, an infrared temperature sensor, an image acquiring device, a pulse detecting instrument and a brain wave acquiring device, so as to acquire the user's temperature information, image information, pulse information and/or brain wave information. The user wearable device may include, but not limited to, a smart bracelet or a smart watch.

The judging module 112 may, according to the user's physiological information acquired by acquiring module 111, identify the user's state as one of the following states: exciting state, tired state, sleepy state and sleeping state. The operations of the judging module 112 will be exemplarily illustrated by taking brain wave information as an example of the user's physiological information.

The judging module 112 may, according to the proportions which various brain electrical activity modes account for in an EEG, identify the user's state as one of the following states: exciting state, tired state, sleepy state, and sleeping state. Specifically, the judging module 112 may identify the user's state as the exciting state when brain wave having frequency of 15 Hz to 30 Hz is dominant; the judging module 112 may identify the user's state as the tired state when brain wave having frequency of 8 Hz to 14 Hz is dominant; the judging module 112 may identify the user's state as the sleepy state when brain wave having frequency of 4 Hz to 7 Hz is dominant; and the judging module 112 may identify the user's state as the sleeping state when brain wave having frequency of 0.5 Hz to 3 Hz is dominant.

Although the operations of the judging module 112 are illustrated by taking brain wave information as an example of the user's physiological information, it should be recognized by those skilled in the art that, the user's state may also be identified by using various kinds of the user's physiological information (for example, temperature information, image information and/or pulse information of the user), and the user's state may, according to the corresponding judging criteria, be identified as one of the following states: exciting state, tired state, sleepy state and sleeping state.

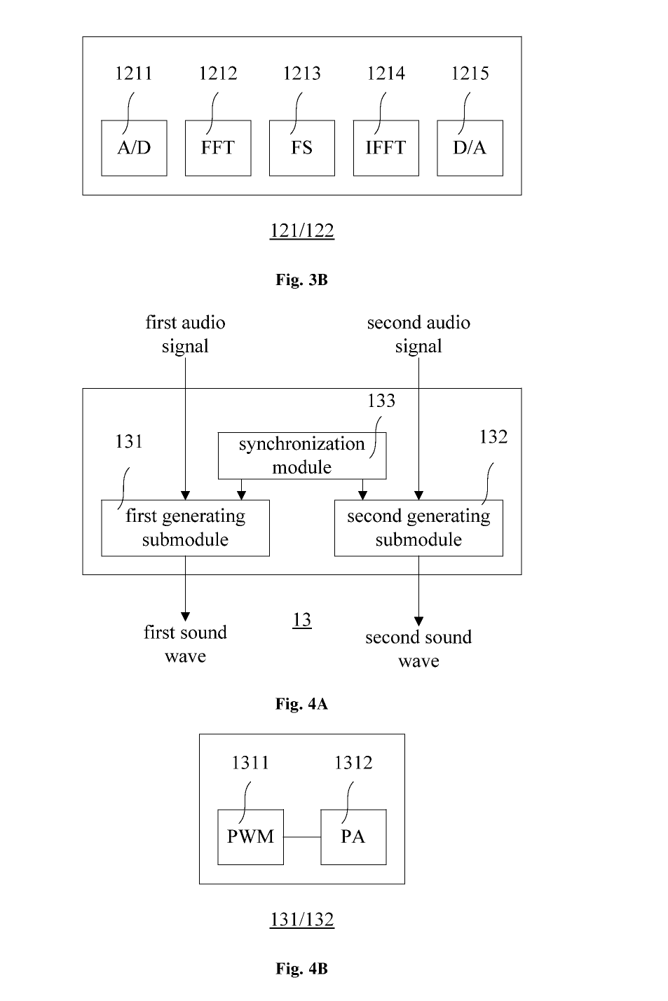

FIG. 3A is a schematic block diagram of the frequency adjusting module 12 included in the audio play device 10, and FIG. 3B is a schematic block diagram of adjusting submodules 121 and 122 included in the frequency adjusting module 12.

Referring to FIG. 3A, the frequency adjusting module 12 may include a first adjusting submodule 121 and a second adjusting submodule 122. The first adjusting submodule 121 and the second adjusting submodule 122 adjust an audio signal from a program source, which is the initial audio signal, according to the user's state identified by the identifying module 11, so as to generate the first audio signal and the second audio signal, respectively.

The first adjusting submodule 121 and the second adjusting submodule 122 of the frequency adjusting module 12 can cause the frequency difference between the first audio signal and the second audio signal to be in a range from 8 Hz to 14 Hz when the user's state is identified as the exiting state by the identifying module 11; the first adjusting submodule 121 and the second adjusting submodule 122 of the frequency adjusting module 12 can cause the frequency difference between the first audio signal and the second audio signal to be in a range from 4 Hz to 7 Hz when the user's state is identified as the tired state by the identifying module 11; and the first adjusting submodule 121 and the second adjusting submodule 122 of the frequency adjusting module 12 can cause the frequency difference between the first audio signal and the second audio signal to be in a range from 0.5 Hz to 3 Hz when the user's state is identified as the sleepy state or sleeping state by the identifying module 11.

The operations of first adjusting submodule 121 and the second adjusting submodule 122 will be described below in details with reference to an embodiment of the present invention. The initial audio signal provided to the frequency adjusting module 12 is transmitted equally to the first adjusting submodule 121 and the second adjusting submodule 122, respectively. One of the first adjusting submodule 121 and the second adjusting submodule 122 (for example, the first adjusting submodule 121) can keep the generated audio signal (for example, the first audio signal) being the same as the initial audio signal, namely keep the frequency of the generated audio signal being the same as that of the initial audio signal. The other of the first adjusting submodule 121 and the second adjusting submodule 122 (for example, the second adjusting submodule 122) can generate an audio signal having a certain frequency difference from the initial audio signal (for example, the second audio signal). FIG. 3B shows the specific operation.

Referring to FIG. 3B, the adjusting submodule for generating the audio signal having frequency difference may include an analog-to-digital converter (A/D) 1211, a fast Fourier transformer (FFT) 1212, a frequency shifter (FS) 1213, an inverse fast Fourier transformer (IFFT) 1214 and a digital-to-analog converter (D/A) 1215. The FS 1213 of the adjusting submodule adjusts the frequency of the audio signal subjected to an A/D conversion and transformed from a time domain to a frequency domain, so as to cause the adjusted frequency has a certain difference from the frequency of the initial audio signal. The adjustment amount of the frequency may be set according to the user's state identified by the identifying module 11. According to an embodiment of the present invention, the adjustment amount of the frequency may be set as 8 Hz to 14 Hz when the user's state is identified as the exiting state by the identifying module 11; the adjustment amount of the frequency may be set as 4 Hz to 7 Hz when the user's state is identified as the tired state by the identifying module 11; and the adjustment amount of the frequency may be set as 0.5 Hz to 3 Hz when the user's state is identified as the sleepy state or sleeping state by the identifying module 11. Then, the output signal of the FS 1213 is subjected to IFFT and D/A conversion, and then the audio signal after frequency adjustment can be output.

It should be realized that the above-mentioned embodiment is merely a preferred embodiment of the present invention. Alternatively, the first adjusting submodule 121 and the second adjusting submodule 122 can adjust the frequency of the initial audio signal simultaneously, without keeping one of the first audio signal and second audio signal being the same as the initial audio signal.

FIG. 4A is a schematic block diagram of the frequency generation module 13 included in the audio play device 10, and FIG. 4B is a schematic block diagram of generating submodules 131 and 132 included in the generation module 13.

Referring to FIG. 4A, the generation module 13 may include a first generation generating submodule 131, a second generation generating submodule 132 and a synchronization module 133 for synchronizing operations of the first generation generating submodule 131 and the second generation generating submodule 132.

The first generating submodule 131 and the second generating submodule 132 generate a first sound wave and a second sound wave respectively according to the first audio signal and the second audio signal. FIG. 3B shows an exemplary structure of the first generating submodule 131 and the second generating submodule 132.

Referring to FIG. 4B, each submodule of the generating submodule 13 may include a pulse width modulator (PWM) 1311 and a power amplifier (PA) 1312. Pulse width modulation and amplification are performed on the input first audio signal or second audio signal to generate the first sound wave and the second wave.

It should be realized that the structure of the above-mentioned generating submodule is merely an embodiment of the present invention. The person skilled in the art can also use a variety of ways to form various generating submodules, as long as they can generate a corresponding sound wave from the input audio signal.

The synchronization module 133 is used for synchronizing the operations of the first generating submodule 131 and the second generating submodule 132, so as to generate the first sound wave and the second sound wave simultaneously. The person skilled in the art can also use a variety of specific implementing ways to form the synchronization module 133.

According to embodiments of the present invention, the playing module 14 included in the audio play device 10 may include one of earphones, directional speakers and directional sound boxes, as long as it can cause the first sound wave and the second sound wave generated by the generation module 13 to enter the left ear and right ear of the user respectively at the same time. Preferably, the playing module 14 may be implemented as earphones.

Although the forgoing illustrates the embodiments of the present invention, it should be understood by the person skilled in the art that the submodules included in the above-mentioned modules may be implemented as separate devices, respective submodules may be combined, or the submodules in some module may be implemented in another module. For example, the A/D 1211 and the FFT 1212 in the adjusting submodule of the frequency adjusting module 12 may be formed as separate devices, thus the signals input to the first adjusting submodule 121 and the second adjusting submodule 122 is a frequency domain signal, and the first adjusting submodule 121 and the second adjusting submodule 122 are implemented as a frequency holder and a frequency shifter, respectively. In addition, the PA 1312 in the generating submodule of the generation module 13 may be implemented in the playing module 14. Accordingly, the scope of the present invention is intended to cover various modifications and variations to the present embodiments.

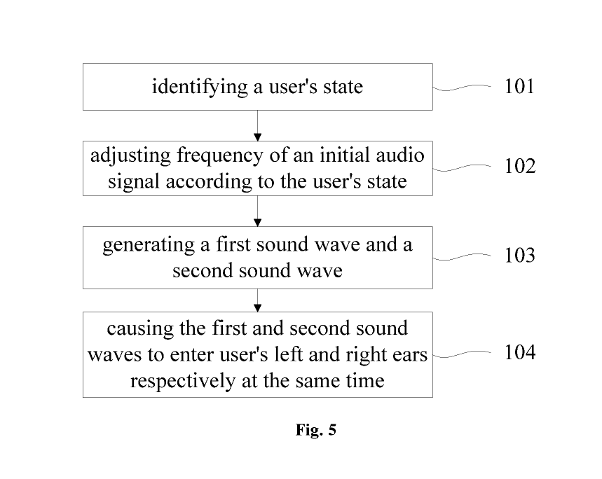

FIG. 5 is a schematic flowchart of an audio play method according to an embodiment of the present invention.

Referring to FIG. 1 and FIG. 5, in step 101, the identifying module 11 of the audio play device 10 identifies a user's state. Specifically, the acquiring module 111 of the identifying module 11 acquires the user's physiological information, and the judging module 112 of the identifying module 11, according to the user's physiological information acquired by the acquiring module 111, identifies the user's state as one of the following states: exciting state, tired state, sleepy state, and sleeping state.

In step 102, the frequency adjusting module 12 of the audio play device 10 adjusts frequency of an initial audio signal according to the user's state identified by the identifying module 11, so as to generate a first audio signal and a second audio signal. Specifically, the frequency adjusting module 12 can cause the frequency difference between the first audio signal and the second audio signal to be in a range from 8 Hz to 14 Hz when the user's state is identified as the exiting state by the identifying module 11; the frequency adjusting module 12 can cause the frequency difference between the first audio signal and the second audio signal to be in a range from 4 Hz to 7 Hz when the user's state is identified as the tired state by the identifying module 11; and the frequency adjusting module 12 can cause the frequency difference between the first audio signal and the second audio signal to be in a range from 0.5 Hz to 3 Hz when the user's state is identified as the sleepy state or sleeping state by the identifying module 11.

In step 103, the generation module 13 of the audio play device 10 generates a first sound wave and a second sound wave respectively according to the first audio signal and the second audio signal. Moreover, in step 104, the playing module 14 of the audio play device 10 causes the first sound wave and the second sound wave to enter the left ear and right ear of the user respectively at the same time.

According to the technical solutions of the present invention, a user's brain electrical activity mode can be changed according to the binaural beats, so as to induce the user to fall asleep. Thus, the user can be induced to fall asleep while the enjoyment of the user on audio and video entertainment is not affected. In addition, an optimum mode can be selected based on the user's state so that the user can fall asleep more effectively in a short time.

The audio play device according to the present invention may be provided separately as an audio player, or combined with a display device with a video play function. For example, the audio play device according to the present invention may be applied in an electronic equipment having video play function, such as a TV set, a mobile phone, a personal computer (PC), a personal digital assistant (PDA), a tablet computer, an game console, a head mounted display (HMD) or the like.

Preferably, the display device may further include a standby module. When the user's state is identified as the sleeping state by the identifying module, the standby module of the display device can disable the display function of the display device to cause it to be in a standby state, thus effectively avoiding unnecessary power consumption.

It should be understood that the above embodiments are merely exemplary embodiments used for illustrating the principle of the present invention, however, the present invention is not limited thereto. Various variations and improvements can be made by the person skilled in the art without departing from the spirit and essence of the present invention, and these variations and improvements should also be considered to be within the protection scope of the present invention.

1. An audio play method for inducing a user to fall asleep, comprising steps of:
identifying the user's state;
adjusting frequency of an initial audio signal according to the identified user's state to generate a first audio signal and a second audio signal having frequency difference, the initial audio signal being an audio signal from a program source;
generating a first sound wave according to the first audio signal and generating a second sound wave according to the second audio signal; and
causing the first sound wave and the second sound wave to enter left ear and right ear of the user respectively at the same time.

2. The audio play method according to claim 1, wherein the step of identifying the user's state comprises:
acquiring the user's physiological information; and
identifying the user's state according to the acquired physiological information.

3. The audio play method according to claim 2, wherein the user's state is, according to the acquired physiological information of the user, identified as one of the following states: exciting state, tired state, sleepy state and sleeping state.

4. The audio play method according to claim 3, wherein the user's physiological information comprises the user's brain wave information, and
according to the brain wave information,
the user's state is identified as the exciting state when brain wave having frequency of 15 Hz to 30 Hz is dominant;
the user's state is identified as the tired state when brain wave having frequency of 8 Hz to 14 Hz is dominant;
the user's state is identified as the sleepy state when brain wave having frequency of 4 Hz to 7 Hz is dominant; and
the user's state is identified as the sleeping state when brain wave having frequency of 0.5 Hz to 3 Hz is dominant.

5. The audio play method according to claim 3, wherein the user's physiological information comprises at least one of temperature information, image information and pulse information.

6. The audio play method according to claim 3, wherein in the step of adjusting the frequency of the initial audio signal according to the user's state to generate the first audio signal and the second audio signal,
causing the frequency difference between the first audio signal and the second audio signal to be in a range from 8 Hz to 14 Hz when the user's state is identified as the exiting state;
causing the frequency difference between the first audio signal and the second audio signal to be in a range from 4 Hz to 7 Hz when the user's state is identified as the tired state;
causing the frequency difference between the first audio signal and the second audio signal to be in a range from 0.5 Hz to 3 Hz when the user's state is identified as the sleepy state or sleeping state.

7. The audio play method according to claim 1, wherein one of the first audio signal and the second audio signal is the same as the initial audio signal.

8. The audio play method according to claim 1, wherein the step of generating the first sound wave according to the first audio signal and generating the second sound wave according to the second audio signal comprises:
performing pulse width modulation on the first audio signal and the second audio signal to form a first pulse width modulation signal and a second pulse width modulation signal respectively; and
amplifying the first pulse width modulation signal and the second pulse width modulation signal to generate the first sound wave and the second wave, respectively.

9. The audio play method according to claim 1, wherein the first audio signal and the second audio signal are generated simultaneously.

10. The audio play method according to claim 1, wherein the first sound wave and the second sound wave are caused to enter the left ear and right ear of the user respectively at the same time by using any one of earphones, directional speakers and directional sound boxes.

11. An audio play device for inducing a user to fall asleep, comprising:
an identifying module for identifying the user's state;
a frequency adjusting module for adjusting frequency of an initial audio signal according to the identified user's state to generate a first audio signal and a second audio signal having frequency difference, the initial audio signal being an audio signal from a program source;
a generation module for generating a first sound wave according to the first audio signal and generating a second sound wave according to the second audio signal; and
a playing module for causing the first sound wave and the second sound wave to enter left ear and right ear of the user respectively at the same time.

12. The audio play device according to claim 11, wherein the identifying module comprises:
an acquiring module for acquiring the user's physiological information; and
a judging module for identifying the user's state according to the acquired physiological information.

13. The audio play device according to claim 12, wherein the judging module identifies, according to the acquired physiological information of the user, the user's state as one of the following states: exciting state, tired state, sleepy state and sleeping state.

14. The audio play device according to claim 13, wherein the acquiring module comprises a brain wave acquiring device for acquiring the user's brain wave information, and
according to the brain wave information,
the judging module identifies the user's state as the exciting state when brain wave having frequency of 15 Hz to 30 Hz is dominant;
the judging module identifies the user's state as the tired state when brain wave having frequency of 8 Hz to 14 Hz is dominant;
the judging module identifies the user's state as the sleepy state when brain wave having frequency of 4 Hz to 7 Hz is dominant; and
the judging module identifies the user's state as the sleeping state when brain wave having frequency of 0.5 Hz to 3 Hz is dominant.

15. The audio play device according to claim 13, wherein the acquiring module comprises at least one of a user wearable device, an infrared temperature sensor, an image acquiring device and a pulse detecting instrument to acquire the temperature information, image information and/or pulse information of the user.

16. The audio play device according to claim 13, wherein
the frequency adjusting module causes the frequency difference between the first audio signal and the second audio signal to be in a range from 8 Hz to 14 Hz when the user's state is identified as the exiting state;
the frequency adjusting module causes the frequency difference between the first audio signal and the second audio signal to be in a range from 4 Hz to 7 Hz when the user's state is identified as the tired state; and
the frequency adjusting module causes the frequency difference between the first audio signal and the second audio signal to be in a range from 0.5 Hz to 3 Hz when the user's state is identified as the sleepy state or sleeping state.

17. The audio play device according to claim 11, wherein the frequency adjusting module keeps one of the first audio signal and the second audio signal being the same as the initial audio signal.

18. The audio play device according to claim 11, wherein
the frequency adjusting module comprises a first adjusting submodule and a second adjusting submodule for adjusting the frequency of the initial audio signal according to the user's state, so as to generate the first audio signal and the second audio signal respectively, and
the generation module comprises a first generating submodule and a second generating submodule for generating the first sound wave and the second sound wave respectively according to the first audio signal and the second audio signal.

19. The audio play device according to claim 18, wherein each of the first generating submodule and the second generating submodule comprises:
a pulse width modulation (PWM) module for performing pulse width modulation on the first audio signal or the second audio signal to form a first pulse width modulation signal or a second pulse width modulation signal; and
a power amplifying module for amplifying the first pulse width modulation signal or the second pulse width modulation signal to generate the first sound wave and the second wave.

20. The audio play device according to claim 18, wherein the generation module further comprises a synchronization module for controlling the first audio signal and the second audio signal to be generated simultaneously.

21. (canceled)

22. (canceled)
